(19) United States
(12) Patent Application Publication
(10) Pub. No.: US 2026/0115044 A1
(43) Pub. Date: Apr. 30, 2026

(54) OPHTHALMIC COMPOSITIONS AND METHODS FOR SUSTAINED DRUG RELEASE

(71) Applicant: EXIMORE LTD., Jerusalem (IL)

(72) Inventors: Eyal SHEETRIT, Jerusalem (IL); Guy TOMER, Modiin (IL)

(21) Appl. No.: 19/117,205

(22) PCT Filed: Sep. 28, 2023
(86) PCT No.: PCT/IB2023/000577

§ 371 Date: Mar. 31, 2025

Related U.S. Application Data
(60) Provisional application No. 63/411,539, filed on Sep. 29, 2022.

(51) Int. Cl. A61F 9/007; A61K 9/00; A61K 9/16; A61K 31/436

(52) U.S. Cl. CPC A61F 9/00772; A61K 9/0048; A61K 9/1611; A61K 9/1641; A61K 9/1652; A61K 31/436

(57) ABSTRACT

Embodiments of the disclosure provide ophthalmic compositions and methods of use thereof. The embodiments disclose ophthalmic compositions including devices such as a punctal plug. Some embodiments are directed to a method of preparing and using such devices. One embodiment provides a method of dissolving a hydrophilic polymer and an active pharmaceutical ingredient (API) in alcohol to form a hydrophilic polymer/API solution, where the API includes tacrolimus, spray drying the hydrophilic polymer/API solution to form a hydrophilic polymer/API mixture, mixing the hydrophilic polymer/API mixture with a binder to form an API paste, and curing the API paste to form a punctal plug, where the punctal plug is configured to release an effective amount of the API for a treatment period of 30 days to one year.

### CROSS REFERENCE TO RELATED APPLICATIONS

This application is an International Application, which claims priority under 35 U.S.C. § 119 (e) to U.S. Provisional Patent Application No. 63/411,539, filed Sep. 29, 2022, which is incorporated herein by reference in its entirety for all purposes.

### TECHNICAL FIELD

In some embodiments, the present disclosure relates to compositions and methods for delivering a bio-active agent or bio-active agents.

### BACKGROUND

The human eye is a complex organ with intricate anatomical and physiological barriers. The anterior segment of the eye consists of the cornea, conjunctiva, aqueous humor, iris, ciliary body, and lens. Common diseases affecting the anterior segment of the eye are Keratoconjunctivitis sicca (KCS), glaucoma, allergic conjunctivitis, anterior uveitis, and cataract.

KCS, also known as dry eye syndrome, is a chronic ophthalmic disease resulting from deficiency of one or more elements in the precorneal tear film. About two percent of the population over 50 years of age suffers from KCS. Common symptoms of KCS include decreased tear production or inadequate secretion of tears, and excessive tear evaporation.

Treatment of KCS and other diseases over time can help to alleviate these symptoms.

### SUMMARY OF THE INVENTION

Embodiments of the present disclosure relate to a method including dissolving a hydrophilic polymer and an active pharmaceutical ingredient (API) in alcohol to form a hydrophilic polymer/API solution, where the API includes tacrolimus. The method also includes spray drying the hydrophilic polymer/API solution to form a hydrophilic polymer/API mixture. The method also includes mixing the hydrophilic polymer/API mixture with a binder to form an API paste. The method also includes curing the API paste to form a punctal plug, where the punctal plug is configured to release an effective amount of the API for a treatment period of 30 days to one year.

In some embodiments, the hydrophilic polymer is hydroxypropyl methylcellulose (HPMC).

In some embodiments, the binder is an epoxy resin.

In some embodiments, the effective amount of the API is at least 0.6 μg per day.

In some embodiments, the effective amount of the API measures from 10% to 30% by weight of a total amount of API mixture.

In some embodiments, the effective amount of the API measures from 30 μg to 90 μg over the treatment period of 30 days to one year.

In some embodiments, the method further includes mixing the hydrophilic polymer/API mixture with at least one additive.

In some embodiments, the at least one additive includes kaolin or polyethylene glycol (PEG).

In some embodiments, the alcohol is ethanol.

Embodiments of the present disclosure also relate to a device including a spray dried composite material including a hydrophilic polymer and an active pharmaceutical ingredient (API), where the API includes tacrolimus. The spray dried composite material also includes a binder. The API has a release rate of at least 0.6 μg per day for a treatment period of 30 days to one year. The device is a punctal plug.

In some embodiments, the hydrophilic polymer is hydroxypropyl methylcellulose (HPMC).

In some embodiments, the binder is an epoxy resin or epoxy.

In some embodiments, the device includes 10% to 40% of tacrolimus by weight.

In some embodiments, the device includes 10% to 40% of the hydrophilic polymer by weight.

In some embodiments, the device includes 30% to 40% of the binder by weight.

In some embodiments, the device further includes an additive.

In some embodiments, the additive includes at least one of kaolin or polyethylene glycol (PEG).

In some embodiments, the device includes 5% to 15% of PEG by weight.

In some embodiments, the device includes 0% to 10% of kaolin by weight.

Embodiments of the present disclosure also relate to a formulation including a spray dried composite material including a hydrophilic polymer and an active pharmaceutical ingredient (API), where the API includes tacrolimus. The formulation also includes a binder. The API has a release rate of at least 0.6 μg per day for a treatment period of 30 days to one year.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

FIG. 1 is a graph depicting accumulated percentage total mass of tacrolimus released, over a 15-day period, for punctal plugs containing tacrolimus, according to embodiments of the present disclosure.

FIG. 2 is a graph depicting accumulated percentage total mass of tacrolimus released, over a 30-day period, for punctal plugs containing tacrolimus, according to embodiments of the present disclosure.

FIG. 3 is a graph depicting the daily release of tacrolimus, over a 30-day period, for punctal plugs containing tacrolimus, according to embodiments of the present disclosure.

### DETAILED DESCRIPTION

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a.” “an,” and “the” include plural references. The meaning of “in” includes “in” and “on.”

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” Spatial or directional terms, such as “left”, “right”, “inner”, “outer”, “above”, “below”, and the like, are not to be considered as limiting as the invention can assume various alternative orientations. All numbers used in the specification are to be understood as being modified in all instances by the term “about”. The term “about” means a range of plus or minus ten percent of the stated value

Unless otherwise indicated, all ranges or ratios disclosed herein are to be understood to encompass any and all subranges or sub-ratios subsumed therein. Unless otherwise indicated, all ranges or ratios herein are understood to be inclusive (i.e., to include both the minimum and maximum values of such ranges or ratios). For example, a stated range or ratio of “1 to 10” should be considered to include any and all subranges between (and inclusive of) the minimum value of 1 and the maximum value of 10; that is, all subranges or sub-ratios beginning with a minimum value of 1 or more and ending with a maximum value of 10 or less, such as but not limited to, 1 to 6.1, 3.5 to 7.8, and 5.5 to 10.

The present disclosure relates generally to the field of medicine combining drugs in a device for administering a bio-active agent over a prolonged period of time. More particularly, it concerns implantable ocular devices for the sustained delivery of a therapeutic compound to the eye. As defined herein, “sustained release” or “sustained delivery” is a type of dosage form which is designed to release a drug at a predetermined rate to maintain a substantially constant drug concentration for a specific period of time with minimum systemic side effects.

While punctal plugs have been a mainstay of therapy for various eye conditions. However, recent studies indicate that inflammation plays a key role in the pathogenesis for many of these conditions. Best et al. (“Punctal and canicular plugs: indications, efficacy and safety), which is hereby incorporated by reference, discloses that punctal plugs may worsen dry eye symptoms if inflammation is not treated prior to insertion of the punctal plug. Further, as disclosed by Lee (“Back to the Shelf for Punctal Plugs), which is hereby incorporated by reference, inserting a punctal plug when there is active inflammation causes blocking of tears/drainage, resulting in an accumulation of cytokines and inflammatory-inducing factors. Thus, as described by both references, if there is active inflammation in the eye, the standard method of care has been to treat inflammation prior to placing punctal plugs.

In some embodiments, the present disclosure relates to punctal plugs that are suitable for placement within the tear duct of the human eye, and which contain therein ophthalmic active pharmaceutical ingredients (APIs) for sustained delivery to the eye. The exemplary embodiments also relate to methods for producing punctal plugs containing ophthalmic APIs therein.

In some embodiments, the exemplary punctal plugs may be used to treat various diseases including, but not limited to, chronic dry eye disease such as: aqueous deficient dry eye, including Sjogren syndrome, evaporative dry eye, including Meibomian gland syndrome and vernal keratoconjunctivitis, and episodic dry eye disease due to cataract and refractive eye surgeries, environmental causes, etc. In some embodiments, a method for producing punctal plugs including an API includes dissolving a hydrophilic polymer and an API in an alcohol to produce a polymer/API solution; spray drying the API/polymer solution to produce a dried API/polymer mixture; optionally milling the dried API/polymer mixture to produce a milled API/polymer mixture; mixing the milled API/polymer mixture with a binder to produce a paste; placing the paste in a mold; and curing the molded paste to produce punctal plugs.

Without being bound by theory, spray drying the API/polymer solution to produce a dried API/polymer mixture converts the API from crystalline to amorphic form, possibly due to the fast cooling of the API/polymer solution. The spray drying of the API/polymer mixture increases the API water solubility and as a result API release rate and extends the API release time when compared to API/polymer mixtures that are dried by other methods including, but not limited to, vacuum drying and ambient conditions drying.

In some embodiments, the method includes mixing the milled API/polymer mixture with a filler or additive.

In some embodiments, the hydrophilic polymer is a hydrophilic cellulose ether derivative. In some embodiments, the hydrophilic polymer is hydroxypropyl methylcellulose (HPMC). In some embodiments, the hydrophilic polymer is methyl cellulose. In some embodiments, the hydrophilic polymer is hydroxy propyl cellulose. In some embodiments, co-spray drying the API with HPMC showed an increased dissolution enhancement effect when compared to other hydrophilic polymers, such as polyvinylpyrrolidone (PVP).

In some embodiments, the exemplary punctal plug includes HPMC at a quantity of 10% to 40% by weight (w/w). In additional embodiments, the exemplary punctal plug described here contains HPMC at a quantity of 10% by weight (w/w) or greater (e.g., 12%, 14%, 16%, 18%, 20%, 22%, 24%, 26%, 28%, 30%, 32%, 34%, 36%, 38%, 40%, 42%, 44%, 46%, 48%, 50%); 50% w/w or less (e.g., 49%, 47%, 45%, 43%, 41%, 39%, 37%, 35%, 33%, 31%, 29%, 27%, 25%, 23%, 21%, 19%, 17%, 15%, 13%, 11%, 9%, 7%, 5%); or 10% w/w-50% w/w (e.g., 11% w/w-49% w/w; 12% w/w-48% w/w; 13% w/w-47% w/w; 14% w/w-46% w/w; 15% w/w-45% w/w; 16% w/w-44% w/w; 17% w/w-43% w/w; 18% w/w-42% w/w; 19% w/w-41% w/w; 20% w/w-40% w/w; 21% w/w-39% w/w; 22% w/w-38% w/w; 23% w/w-37% w/w; 24% w/w-36% w/w; 25% w/w-35% w/w; 26% w/w-34% w/w; 27% w/w-33% w/w; 28% w/w-32% w/w; 29% w/w-31% w/w). In some embodiments, the exemplary punctal plug includes HPMC at a quantity of 15% to 40% by weight. In some embodiments, the exemplary punctal plug includes HPMC at a quantity of 20% to 40% by weight. In some embodiments, the exemplary punctal plug includes HPMC at a quantity of 25% to 40% by weight. In some embodiments, the exemplary punctal plug includes HPMC at a quantity of 30% to 40% by weight. In some embodiments, the exemplary punctal plug includes HPMC at a quantity of 35% to 40% by weight.

In some embodiments, the exemplary punctal plug includes HPMC at a quantity of 10% to 35% by weight. In some embodiments, the exemplary punctal plug includes HPMC at a quantity of 10% to 30% by weight. In some embodiments, the exemplary punctal plug includes HPMC at a quantity of 10% to 25% by weight. In some embodiments, the exemplary punctal plug includes HPMC at a quantity of 10% to 20% by weight. In some embodiments, the exemplary punctal plug includes HPMC at a quantity of 10% to 15% by weight.

In some embodiments, the exemplary punctal plug includes HPMC at a quantity of 12% to 22% by weight. In some embodiments, the exemplary punctal plug includes HPMC at a quantity of 18% to 27% by weight. In some embodiments, the exemplary punctal plug includes HPMC at a quantity of 21% to 25% by weight. In some embodiments, the exemplary punctal plug includes HPMC at a quantity of 32% to 38% by weight. In some embodiments, the exemplary punctal plug includes HPMC at a quantity of 14% to 36% by weight.

In some embodiments, the hydrophilic polymer is not polyvinylpyrrolidone (PVP).

In some embodiments, the API is an immunosuppressive drug. In some embodiments, the API is an immunosuppressive drug that is an antibiotic macrolide. In some embodiments, the API is tacrolimus. In some embodiments, the API includes cyclosporine A, prednisolone acetate, dexamethasone, ibuprofen, tobramycin, moxifloxacin, gatifloxacin, latanoprost, travaprost, timolol, atropine or combinations thereof.

In some embodiments, tacrolimus (FK-506), an antibiotic macrolide derived from the bacterium Streptomyces tsukubaensis, is a potent immunomodulator capable of decreasing the production of inflammatory mediators by T lymphocytes through the inhibition of calcineurin, an intracytoplasmic protein essential for interleukin (IL)-2 and IL-4 transcription.

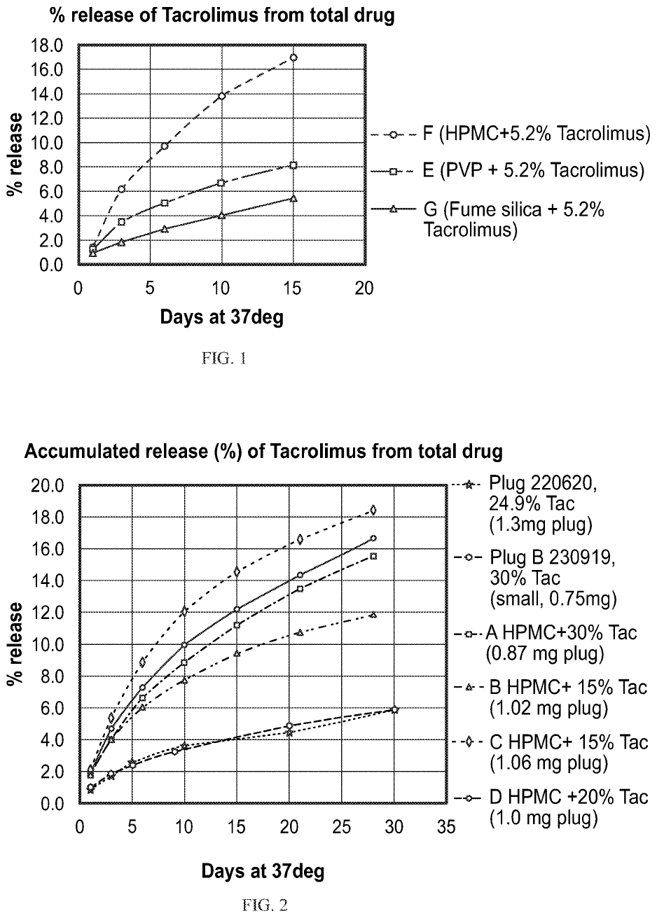

Tacrolimus (IUPAC name: (1R,9S,12S,13R,14S,17R,18E,21S,23S,24R,25S,27R)-1,14-dihydroxy-12-[(E)-1-[(1R,3R,4R)-4-hydroxy-3-methoxycyclohexyl]prop-1-en-2-yl]-23,25-dimethoxy-13,19,21,27-tetramethyl-17-prop-2-enyl-11,28-dioxa-4-azatricyclo[22.3.1.04,9]octacos-18-ene-2,3,10,16-tetrone: hydrate) also referred to as FK-506, FR-900506, and Fujimycin, is a macrolide isolated from Streptomyces tsukubaensis having the chemical structure illustrated in FIG. 1.

Tacrolimus binds to the FKBP-12 protein and forms a complex with calcium-dependent proteins, thereby inhibiting calcineurin phosphatase activity and resulting in decreased cytokine production. This agent exhibits potent immunosuppressive activity in vivo and prevents the activation of T-lymphocytes in response to antigenic or mitogenic stimulation.

Tacrolimus is also effective in the treatment of immune-mediated diseases such as corneal graft rejection, ocular inflammation, ocular pemphigoid, allergic rhinitis, and uveitis.

In some embodiments, the exemplary punctal plug includes tacrolimus at a quantity of 10% to 40% by weight. In some embodiments, the exemplary punctal plug includes tacrolimus at a quantity of 15% to 40% by weight. In some embodiments, the exemplary punctal plug includes tacrolimus at a quantity of 20% to 40% by weight. In some embodiments, the exemplary punctal plug includes tacrolimus at a quantity of 25% to 40% by weight. In some embodiments, the exemplary punctal plug includes tacrolimus at a quantity of 30% to 40% by weight. In some embodiments, the exemplary punctal plug includes tacrolimus at a quantity of 35% to 40% by weight.

In some embodiments, the exemplary punctal plug includes tacrolimus at a quantity of 10% to 35% by weight. In additional embodiments, the exemplary punctal plug described here contains an active ingredient, such as but not limited to, tacrolimus, which is present in the punctal plug in an amount of 10% by weight (w/w) or greater (e.g., 12%, 14%, 16%, 18%, 20%, 22%, 24%, 26%, 28%, 30%, 32%, 34%, 36%, 38%, 40%, 42%, 44%, 46%, 48%, 50%); 50% w/w or less (e.g., 49%, 47%, 45%, 43%, 41%, 39%, 37%, 35%, 33%, 31%, 29%, 27%, 25%, 23%, 21%, 19%, 17%, 15%, 13%, 11%, 9%, 7%, 5%); or 10% w/w-50% w/w (e.g., 11% w/w-49% w/w; 12% w/w-48% w/w; 13% w/w-47% w/w; 14% w/w-46% w/w; 15% w/w-45% w/w; 16% w/w-44% w/w; 17% w/w-43% w/w; 18% w/w-42% w/w; 19% w/w-41% w/w; 20% w/w-40% w/w; 21% w/w-39% w/w; 22% w/w-38% w/w; 23% w/w-37% w/w; 24% w/w-36% w/w; 25% w/w-35% w/w; 26% w/w-34% w/w; 27% w/w-33% w/w; 28% w/w-32% w/w; 29% w/w-31% w/w). In some embodiments, the exemplary punctal plug includes tacrolimus at a quantity of 10% to 30% by weight. In some embodiments, the exemplary punctal plug includes tacrolimus at a quantity of 10% to 25% by weight. In some embodiments, the exemplary punctal plug includes tacrolimus at a quantity of 10% to 20% by weight. In some embodiments, the exemplary punctal plug includes tacrolimus at a quantity of 10% to 15% by weight.

In some embodiments, the exemplary punctal plug includes tacrolimus at a quantity of 12% to 22% by weight. In some embodiments, the exemplary punctal plug includes tacrolimus at a quantity of 18% to 27% by weight. In some embodiments, the exemplary punctal plug includes tacrolimus at a quantity of 21% to 25% by weight. In some embodiments, the exemplary punctal plug includes tacrolimus at a quantity of 32% to 38% by weight. In some embodiments, the exemplary punctal plug includes tacrolimus at a quantity of 14% to 36% by weight.

In some embodiments, the ratio of HPMC to tacrolimus is 1:2.5. In some embodiments, the ratio of HPMC to tacrolimus is 1:4 to 4:1. In some embodiments, the ratio of HPMC to tacrolimus is 1:2 to 4:1. In some embodiments, the ratio of HPMC to tacrolimus is 1:1 to 4:1. In some embodiments, the ratio of HPMC to tacrolimus is 2:1 to 4:1. In some embodiments, the ratio of HPMC to tacrolimus is 3:1 to 4:1.

In some embodiments, the ratio of HPMC to tacrolimus is 1:3 to 4:1. In some embodiments, the ratio of HPMC to tacrolimus is 1:2 to 4:1. In some embodiments, the ratio of HPMC to tacrolimus is 1:1 to 4:1. In some embodiments, the ratio of HPMC to tacrolimus is 2:1 to 4:1. In some embodiments, the ratio of HPMC to tacrolimus is 3:1 to 4:1.

In some embodiments, the ratio of HPMC to tacrolimus is 1:1 to 3:1. In some embodiments, the ratio of HPMC to tacrolimus is 1:3 to 2:1. In some embodiments, the ratio of HPMC to tacrolimus is 1:2 to 1:1. In some embodiments, the ratio of HPMC to tacrolimus is 1:2 to 2:1.

In some embodiments, the binder is an epoxy resin. While epoxy resin is not safe for oral use, it was found that epoxy resin is an effective binder for a punctal plug, increasing the release rate and extending the release time of the API in the punctal plug.

In additional embodiments, the exemplary punctal plug described here contains a binder, such as but not limited to, epoxy, which is present in the punctal plug in an amount of 10% by weight (w/w) or greater (e.g., 12%, 14%, 16%, 18%, 20%, 22%, 24%, 26%, 28%, 30%, 32%, 34%, 36%, 38%, 40%, 42%, 44%, 46%, 48%, 50%); 50% w/w or less (e.g., 49%, 47%, 45%, 43%, 41%, 39%, 37%, 35%, 33%, 31%, 29%, 27%, 25%, 23%, 21%, 19%, 17%, 15%, 13%, 11%, 9%, 7%, 5%); or 10% w/w-50% w/w (e.g., 11% w/w-49% w/w; 12% w/w-48% w/w; 13% w/w-47% w/w; 14% w/w-46% w/w; 15% w/w-45% w/w; 16% w/w-44% w/w; 17% w/w-43% w/w; 18% w/w-42% w/w; 19% w/w-41% w/w; 20% w/w-40% w/w; 21% w/w-39% w/w; 22% w/w-38% w/w; 23% w/w-37% w/w; 24% w/w-36% w/w; 25% w/w-35% w/w; 26% w/w-34% w/w; 27% w/w-33% w/w; 28% w/w-32% w/w; 29% w/w-31% w/w). In some embodiments, the exemplary punctal plug includes an epoxy at a quantity of 30% to 40% by weight. In some embodiments, the exemplary punctal plug includes an epoxy at a quantity of 32% to 40% by weight. In some embodiments, the exemplary punctal plug includes an epoxy at a quantity of 34% to 40% by weight. In some embodiments, the exemplary punctal plug includes an epoxy at a quantity of 36% to 40% by weight. In some embodiments, the exemplary punctal plug includes an epoxy at a quantity of 38% to 40% by weight.

In some embodiments, the exemplary punctal plug includes an epoxy at a quantity of 30% to 38% by weight. In some embodiments, the exemplary punctal plug includes an epoxy at a quantity of 30% to 36% by weight. In some embodiments, the exemplary punctal plug includes an epoxy at a quantity of 30% to 34% by weight. In some embodiments, the exemplary punctal plug includes an epoxy at a quantity of 30% to 32% by weight.

In some embodiments, the exemplary punctal plug includes an epoxy at a quantity of 32% to 36% by weight. In some embodiments, the exemplary punctal plug includes an epoxy at a quantity of 31% to 33% by weight. In some embodiments, the exemplary punctal plug includes an epoxy at a quantity of 34% to 37% by weight. In some embodiments, the exemplary punctal plug includes an epoxy at a quantity of 35% to 39% by weight. In some embodiments, the exemplary punctal plug includes an epoxy at a quantity of 31% to 39% by weight.

In some embodiments, the alcohol is ethanol.

In some embodiments, an exemplary punctal plug includes at least one additive. In some embodiments, the at least one additive includes a biocompatible plasticizer included so as to make the punctal plug more flexible and less brittle, to facilitate its removal from the mold intact, and to reduce the risk of injuries to the punctum and lacrimal canal tissues as a result of a tough and non-flexible biomaterial. In some embodiments, the plasticizer includes polyethylene glycol (PEG). In some embodiments, the plasticizer includes PEG 400.

In some embodiments, the exemplary punctal plug includes PEG 400 at a quantity of 5% to 15%. In additional embodiments, the exemplary punctal plug described here contains a plasticizer, such as but not limited to, PEG 400, which is present in the punctal plug in an amount of 1% by weight (w/w) or greater (e.g., 2%, 4%, 6%, 8%, 10%, 12%, 14%, 16%, 18%, 20%); 20% w/w or less (e.g., 19%, 17%, 15%, 13%, 11%, 9%, 7%, 5%, 3%, 1%); or 1% w/w-20% w/w (e.g., 2% w/w-19% w/w; 3% w/w-18% w/w; 4% w/w-17% w/w; 5% w/w-16% w/w; 6% w/w-15% w/w; 7% w/w-14% w/w; 8% w/w-13% w/w; 9% w/w-12% w/w; 10% w/w-11% w/w). In some embodiments, the exemplary punctal plug includes PEG 400 at a quantity of 7% to 15%. In some embodiments, the exemplary punctal plug includes PEG 400 at a quantity of 9% to 15%. In some embodiments, the exemplary punctal plug includes PEG 400 at a quantity of 11% to 15%. In some embodiments, the exemplary punctal plug includes PEG 400 at a quantity of 13% to 15%.

In some embodiments, the exemplary punctal plug includes PEG 400 at a quantity of 5% to 13%. In some embodiments, the exemplary punctal plug includes PEG 400 at a quantity of 5% to 11%. In some embodiments, the exemplary punctal plug includes PEG 400 at a quantity of 5% to 9%. In some embodiments, the exemplary punctal plug includes PEG 400 at a quantity of 5% to 7%.

In some embodiments, the exemplary punctal plug includes PEG 400 at a quantity of 6% to 14%. In some embodiments, the exemplary punctal plug includes PEG 400 at a quantity of 7% to 12%. In some embodiments, the exemplary punctal plug includes PEG 400 at a quantity of 9% to 13%. In some embodiments, the exemplary punctal plug includes PEG 400 at a quantity of 11% to 14%. In some embodiments, the exemplary punctal plug includes PEG 400 at a quantity of 8% to 12%.

In some embodiments, the filler is an absorbent, such as kaolin. In some embodiments, the exemplary punctal plug includes kaolin at a quantity of 1% to 10% by weight. In additional embodiments, the exemplary punctal plug described here contains a filler, such as but not limited to, kaolin, which is present in the punctal plug in an amount of 1% by weight (w/w) or greater (e.g., 2%, 4%, 6%, 8%, 10%, 12%, 14%, 16%, 18%, 20%); 20% w/w or less (e.g., 19%, 17%, 15%, 13%, 11%, 9%, 7%, 5%); or 1% w/w-20% w/w (e.g., 2% w/w-19% w/w; 3% w/w-18% w/w; 4% w/w-17% w/w; 5% w/w-16% w/w; 6% w/w-15% w/w; 7% w/w-14% w/w; 8% w/w-13% w/w; 9% w/w-12% w/w; 10% w/w-11% w/w). In some embodiments, the exemplary punctal plug includes kaolin at a quantity of 1% to 8% by weight. In some embodiments, the exemplary punctal plug includes kaolin at a quantity of 1% to 6% by weight. In some embodiments, the exemplary punctal plug includes kaolin at a quantity of 1% to 4% by weight. In some embodiments, the exemplary punctal plug includes kaolin at a quantity of 1% to 2% by weight.

In some embodiments, the exemplary punctal plug includes kaolin at a quantity of 2% to 10% by weight. In some embodiments, the exemplary punctal plug includes kaolin at a quantity of 4% to 10% by weight. In some embodiments, the exemplary punctal plug includes kaolin at a quantity of 6% to 10% by weight. In some embodiments, the exemplary punctal plug includes kaolin at a quantity of 8% to 10% by weight.

In some embodiments, the exemplary punctal plug includes kaolin at a quantity of 2% to 6% by weight. In some embodiments, the exemplary punctal plug includes kaolin at a quantity of 4% to 8% by weight. In some embodiments, the exemplary punctal plug includes kaolin at a quantity of 6% to 9% by weight. In some embodiments, the exemplary punctal plug includes kaolin at a quantity of 1% to 3% by weight. In some embodiments, the exemplary punctal plug includes kaolin at a quantity of 1% to 5% by weight.

In some embodiments, the release rate of tacrolimus is extended by the process of spray drying the tacrolimus/polymer mixture. That is, spray drying the tacrolimus/polymer mixture, as opposed to other forms of drying including, but not limited to, vacuum drying, ambient pressure drying, was found to extend the period of time over which tacrolimus is released from the punctal plug. In some embodiments of the composition of the present invention, the punctal plug is configured to release tacrolimus over a period of time, for example, for at least two weeks or, for example, for two weeks to one year, after intraocular administration of the tacrolimus containing punctal plug. In some embodiments, the period of time is one month to one year. In some embodiments, the period of time is four months to one year. In some embodiments, the period of time is six months to one year. In some embodiments, the period of time is eight months to one year. In some embodiments, the period of time is ten months to one year.

In some embodiments, the period of time is two weeks to ten months. In some embodiments, the period of time is two weeks to eight months. In some embodiments, the period of time is two weeks to six months. In some embodiments, the period of time is two weeks to four months. In some embodiments, the period of time is two weeks to two months. In some embodiments, the period of time is two weeks to one month.

In some embodiments, the period of time is two months to six months. In some embodiments, the period of time is six months to ten months. In some embodiments, the period of time is one month to eight months. In some embodiments, the period of time is one month to two months. In some embodiments, the period of time is one month to six months. In some embodiments, the period of time is four months to eight months.

In some embodiments, the punctal plug is configured to release at least 0.6 μg of tacrolimus per day for 28 days.

In some embodiments, the punctal plug is configured to cumulatively release 10% to 30% of the total amount of tacrolimus over 28 days. In some embodiments, the punctal plug is configured to cumulatively release 15% to 30% of the total amount of tacrolimus over 28 days. In some embodiments, the punctal plug is configured to cumulatively release 20% to 30% of the total amount of tacrolimus over 28 days. In some embodiments, the punctal plug is configured to cumulatively release 25% to 30% of the total amount of tacrolimus over 28 days.

In some embodiments, the punctal plug is configured to cumulatively release 10% to 25% of the total amount of tacrolimus over 28 days. In some embodiments, the punctal plug is configured to cumulatively release 10% to 20% of the total amount of tacrolimus over 28 days. In some embodiments, the punctal plug is configured to cumulatively release 10% to 15% of the total amount of tacrolimus over 28 days.

In some embodiments, the punctal plug is configured to cumulatively release 15% to 25% of the total amount of tacrolimus over 28 days. In some embodiments, the punctal plug is configured to cumulatively release 15% to 20% of the total amount of tacrolimus over 28 days. In some embodiments, the punctal plug is configured to cumulatively release 20% to 25% of the total amount of tacrolimus over 28 days.

In some embodiments, the punctal plug is configured to cumulatively release 30 μg to 90 μg tacrolimus over 28 days. In some embodiments, the punctal plug is configured to cumulatively release 40 μg to 90 μg tacrolimus over 28 days. In some embodiments, the punctal plug is configured to cumulatively release 50 μg to 90 μg tacrolimus over 28 days. In some embodiments, the punctal plug is configured to cumulatively release 60 μg to 90 μg tacrolimus over 28 days. In some embodiments, the punctal plug is configured to cumulatively release 70 μg to 90 μg tacrolimus over 28 days. In some embodiments, the punctal plug is configured to cumulatively release 80 μg to 90 μg tacrolimus over 28 days.

In some embodiments, the punctal plug is configured to cumulatively release 30 μg to 80 μg tacrolimus over 28 days. In some embodiments, the punctal plug is configured to cumulatively release 30 μg to 70 μg tacrolimus over 28 days. In some embodiments, the punctal plug is configured to cumulatively release 30 μg to 60 μg tacrolimus over 28 days. In some embodiments, the punctal plug is configured to cumulatively release 30 μg to 50 μg tacrolimus over 28 days. In some embodiments, the punctal plug is configured to cumulatively release 30 μg to 40 μg tacrolimus over 28 days.

In some embodiments, the punctal plug is configured to cumulatively release 25 μg to 75 μg tacrolimus over 28 days. In some embodiments, the punctal plug is configured to cumulatively release 40 μg to 60 μg tacrolimus over 28 days. In some embodiments, the punctal plug is configured to cumulatively release 50 μg to 80 μg tacrolimus over 28 days. In some embodiments, the punctal plug is configured to cumulatively release 45 μg to 85 μg tacrolimus over 28 days. In some embodiments, the punctal plug is configured to cumulatively release 70 μg to 80 μg tacrolimus over 28 days. In some embodiments, the punctal plug is configured to cumulatively release 35 μg to 55 μg tacrolimus over 28 days.

In some embodiments, a bad release rate of the punctal plug is less than 0.6 μg for any day within a 28-day release period. In some embodiments, a bad cumulative release rate of the punctal plug is less than 10% or greater than 30% of the total amount of tacrolimus over 28 days. In some embodiments, a bad cumulative release rate of the punctal plug is 30 μg or less or greater than 90 μg.

### Example 1: Preparation of Punctal Plug Including Tacrolimus and PVP

1.524 grams of PVP was dissolved in 38 milliliters of 80% ethanol to form a 4% w/v PVP solution 380 mg of tacrolimus was added to the solution on the day of spray drying to get a 1% w/v tacrolimus solution. The solution was fed into a spray drying machine. The spray drying machine was configured to operate as follows:
Instrument: Mini Buchi B-290 spray dryer
Inlet temperature: 110° C.;
Outlet temperature: 75° C.-78° C.;
Airflow: 30 m³/h
Nozzle: 0.7 mm

The spray dried tacrolimus/PVP mixture was milled using a pestle and mortar. Tacrolimus constituted 20% of the total solids in the finished formulation and PVP constituted 80% of the total solids in the finished formulation.

The tacrolimus/PVP mixture was mixed with PEG 400, kaolin and an epoxy resin until a homogenous paste was achieved.

The paste formulation included 25.9% tacrolimus/PVP mixture by weight, 20% kaolin by weight, 43.8% epoxy resin by weight and 10.3% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 2: Preparation of Punctal Plug Including Tacrolimus and HPMC

1.519 grams of HPMC (Sigma Aldrich 423238, average Mn ˜10,000, viscosity 6 cP (20° C.) (lit.)) were added to 24 grams of 80% ethanol while stirring to make a suspension. 7.6 milliliters of cold water were added to the suspension to form a clear transparent 4% w/v HPMC solution. 380 milligrams of tacrolimus (CCSB (Taiwan)) were added to the solution on the day of spray drying to get a 1% w/v tacrolimus solution. The solution was fed into a spray drying machine. The spray drying machine was configured to operate as discussed with respect to Example 1. The spray dried tacrolimus/HPMC mixture was milled using a pestle and mortar.

Tacrolimus constituted 20% of the total solids in the finished formulation and HPMC constituted 80% of the total solids in the finished formulation.

The tacrolimus/HPMC mixture was mixed with PEG 400, kaolin and an epoxy resin until a homogenous paste was achieved.

The paste formulation included 26.1% tacrolimus/HPMC mixture by weight, 20% Kaolin by weight, 43.7% epoxy resin by weight and 10.3% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 3: Preparation of Punctal Plug Including Tacrolimus and Fume Silica

1.519 grams of Fume Silica were added to 60 grams of 96% ethanol while stirring to make a suspension. 380 milligrams of tacrolimus were added to the solution on the day of spray drying to form a 0.5% w/v tacrolimus solution. The solution was fed into a spray drying machine. The spray drying machine was configured to operate as discussed with respect to Example 1. The dried tacrolimus/fume silica mixture was milled using a pestle and mortar.

Tacrolimus constituted 20% of the total solids in the finished formulation and fume silica constituted 80% of the total solids in the finished formulation.

The tacrolimus/fume silica mixture was mixed with PEG 400, kaolin and an epoxy resin until a homogenous paste was achieved.

The paste formulation included 25.9% tacrolimus/fume silica mixture by weight, 19.2% Kaolin by weight, 45% epoxy resin by weight and 10% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

Table 1 provides the formulations of the punctal plugs of Examples 1 through 3. Tacrolimus (TAC).

TABLE 1

| | | | TAC/ | | | | | | | |
|---|---|---|---|---|---|---|---|---|---|---|
| | TAC/ | TAC/ | Fume | | Epoxy | | | | | Fume |
| Example | PVP | HPMC | silica | Kaolin | resin | PEG400 | TAC | PVP | HPMC | Silica |
| 1 | 25.9% | | | 20% | 43.8% | 10.3% | 5.2% | 20.8% | | |
| 2 | | 26.1% | | 20% | 43.7% | 10.3% | 5.2% | | 20.8% | |
| 3 | | | 25.9% | 19.2% | 45% | 10% | 5.2% | | | 20.8% |

### Example 4: Preparation of Punctal Plug Including Tacrolimus and HPMC

855 milligrams of HPMC were added to 24 grams of 80% ethanol while stirring to make a suspension. 7.65 milliliters of cold water were added to the suspension to form a clear transparent 2.25% w/v HPMC solution. 550.5 milligrams of tacrolimus were added to 36.7 milliliters of the solution on the day of spray drying to get a 1.5% w/v tacrolimus solution. The solution was fed into a spray drying machine. The spray drying machine was configured to operate as discussed with respect to Example 1. The spray dried tacrolimus/HPMC mixture was milled using a pestle and mortar.

Tacrolimus constituted 40% of the total solids in the finished formulation and HPMC constituted 60% of the total solids in the finished formulation.

The tacrolimus/HPMC mixture was mixed with PEG 400, kaolin and an epoxy resin until a homogenous paste was achieved.

The paste formulation included 37.3% tacrolimus/HPMC mixture by weight, 10.1% kaolin by weight, 42.4% epoxy resin by weight and 10.1% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 5: Preparation of Punctal Plug Including Tacrolimus and HPMC

855 milligrams of HPMC were added to 24 grams of 80% ethanol while stirring to make a suspension. 7.65 milliliters of cold water were added to the suspension to form a clear transparent 2.25% w/v HPMC solution. 550.5 milligrams of tacrolimus were added to 36.7 milliliters of the solution on the day of spray drying to get a 1.5% w/v tacrolimus solution. The solution was fed into a spray drying machine. The spray drying machine was configured to operate as discussed with respect to Example 1. The spray dried tacrolimus/HPMC mixture was milled using a pestle and mortar.

Tacrolimus constituted 40% of the total solids in the finished formulation and HPMC constituted 60% of the total solids in the finished formulation.

The tacrolimus/HPMC mixture was mixed with PEG 400 and an epoxy resin until a homogenous paste was achieved.

The paste formulation included 50.1% tacrolimus/HPMC mixture by weight, 40.3% epoxy resin by weight and 9.6% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 6: Preparation of Punctal Plug Including Tacrolimus and HPMC

507 milligrams of HPMC were added to 24 grams of 80% ethanol while stirring to make a suspension. 7.6 milliliters of cold water were added to the suspension to form a clear transparent 1.5% w/v HPMC solution. 740 milligrams of tacrolimus were added to 37 milliliters of the solution on the day of spray drying to get a 2% w/v tacrolimus solution. The solution was fed into a spray drying machine. The spray drying machine was configured to operate as discussed with respect to Example 1. The spray dried tacrolimus/HPMC mixture was milled using a pestle and mortar.

Tacrolimus constituted 60% of the total solids in the finished formulation and HPMC constituted 40% of the total solids in the finished formulation.

The spray dried tacrolimus/HPMC mixture was mixed with PEG 400 and an epoxy resin until a homogenous paste was achieved.

The paste formulation included 50% tacrolimus/HPMC mixture by weight, 40.4% epoxy resin by weight and 9.7% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 7: Preparation of Punctal Plug Including Tacrolimus and HPMC

507 milligrams of HPMC were added to 24 grams of 80% ethanol while stirring to make a suspension. 7.6 milliliters of cold water were added to the suspension to form a clear transparent 1.5% w/v HPMC solution. 740 milligrams of tacrolimus were added to 37 milliliters of the solution on the day of spray drying to get a 2% w/v tacrolimus solution. The solution was fed into a spray drying machine. The spray drying machine was configured to operate as discussed with respect to Example 1. The spray dried tacrolimus/HPMC mixture was milled using a pestle and mortar.

Tacrolimus constituted 60% of the total solids in the finished formulation and HPMC constituted 40% of the total solids in the finished formulation.

The tacrolimus/HPMC mixture was mixed with PEG 400, kaolin and an epoxy resin until a homogenous paste was achieved.

The paste formulation included 25.1% tacrolimus/HPMC mixture by weight, 20.5% kaolin by weight, 43.9% epoxy resin by weight and 10.5% PEG 400 by weight.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

Table 2 provides the formulations of the punctal plugs of Examples 4 through 7.

TABLE 2

| | S.D #10 | S.D#11 | | Epoxy | | | |
|---|---|---|---|---|---|---|---|
| Formulation | (HPMC) | (HPMC) | Kaolin | resin | PEG400 | Tacrolimus | HPMC |
| 4 | 37.3% | | 10.1% | 42.4% | 10.1% | 15% | 22.4% |
| 5 | 50.1% | | | 40.3% | 9.6% | 20% | 30% |
| 6 | | 50% | | 40.4% | 9.7% | 30% | 20% |
| 7 | | 25.1% | 20.5% | 43.9% | 10.5% | 15% | 10% |

Table 3 provides the punctal plugs that were prepared from the above formulations.

TABLE 3

| | | | % Tacro- | | | | |
|---|---|---|---|---|---|---|---|
| Exam- | Plug | | limus | Tacrolimus in plug | | Release medium | |
| ple | weight | | w/w | (theoretical) | | volume | |
| 1 | 1.6 | mg | 5.2% | 83 | μg | 0.5 | ml |
| | 1.8 | mg | 5.2% | 94 | μg | 5 | ml |
| 2 | 1.3 | mg | 5.2% | 68 | μg | 0.5 | ml |
| | 1.3 | mg | 5.2% | 68 | μg | 0.5 | ml |
| 3 | 1.4 | mg | 5.2% | 73 | μg | 0.5 | ml |
| | 1.4 | mg | 5.2% | 73 | μg | 0.5 | ml |
| 4 | 1.06 | mg | 15% | 159 | μg | 0.25 | ml |
| | 0.98 | mg | 15% | 147 | μg | 0.25 | ml |
| 5 | 1 | mg | 20% | 200 | μg | 0.33 | ml |
| | 0.94 | mg | 20% | 188 | μg | 0.33 | ml |
| 6 | 0.87 | mg | 30% | 261 | μg | 0.5 | ml |
| | 0.53 | mg | 30% | 159 | μg | 0.25 | ml |
| 7 | 1.02 | mg | 15% | 153 | μg | 0.25 | ml |
| | 1.02 | mg | 15% | 153 | μg | 0.25 | ml |

### Example 8: Release Rate of Tacrolimus from Punctal Plugs

A 0.5 mg plug was placed in a microfuge tube containing 0.5 mL of a release medium (0.005% benzalkonium chloride, 0.11% Triton X-100 in PBS). The tube was incubated at 37° C. on an orbital shaker at 50 rpm.

At designated time points occurring after 1, 3, 5, 9, 20, 30, 47, 60 and 93 days, the plugs were transferred from the current tube to a new tube containing the same quantity of fresh release medium and incubation continued under the same conditions.

The supernatants from each sample were injected into an HPLC machine. The HPLC machine was configured to operate as follows:

### HPLC Running Conditions:

Instrument: Waters 2695 separation module coupled to Waters 996 PDA detector
Column: Phenomenex 00G-4601-E0 5μ C18 250*4.6 mm 100 Å
Mobile Phase A: ACN:Water: H₃PO₄=700:300:0.2
Diluent: ACN:Water 50:50
Column temperature: 60° C.+/−5° C.
Sample Temperature: Ambient
Injection volume: 20 μL
Detector: PDA at 200 nm
Standard calibration curve showed linearity between 1 μg/ml to 20 μg/ml

FIG. 1 depicts accumulated percentage total mass of tacrolimus released, over a 15-day period, for punctal plugs containing tacrolimus prepared in accordance with Example 1 (shown in gray) Example 2 (shown in orange) and Example 3 (shown in yellow). It may be seen that the percentage of total mass of tacrolimus released by punctal plugs containing HPMC (Example 2) after 15 days exceeded 16%, while the percentage of total mass of tacrolimus released by punctal plugs containing PVP (Example 1) or fume silica (Example 3) after 15 days was 8% or less.

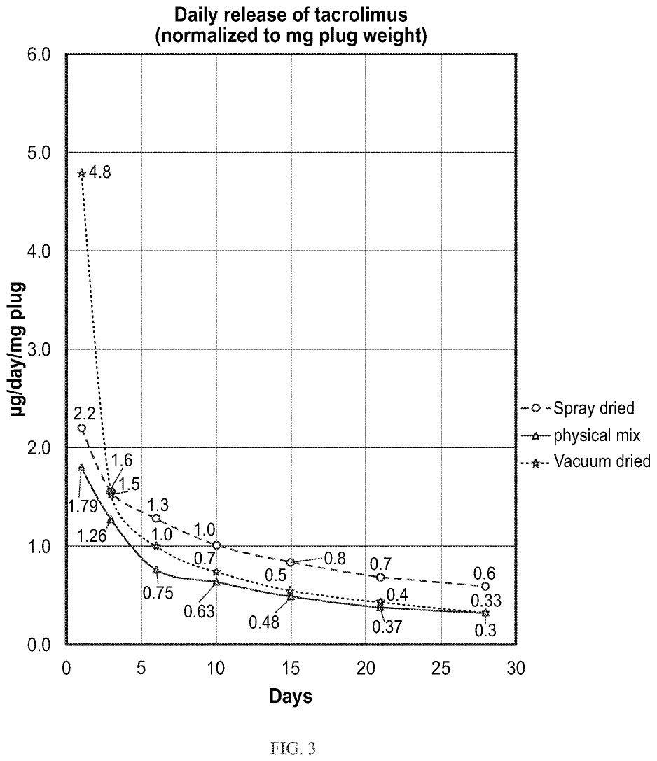

FIG. 2 depicts accumulated percentage total mass of tacrolimus released, over a 30-day period, for punctal plugs containing tacrolimus prepared in accordance with Example 4 (shown in red) Example 5 (shown in gray) and Example 6 (shown in gold) and Example 7 (shown in dark blue).

### Example 9: Preparation of Composite Material and Punctal Plugs Containing Tacrolimus Via Vacuum Drying

507 milligrams of HPMC were added to 24 grams of 80% ethanol while stirring to make a suspension. 7.6 milliliters of cold water were added to the suspension to form a clear transparent HPMC solution. 430 milligrams of tacrolimus were added to 37 milliliters of the solution to get a tacrolimus solution. The 7.06 grams of the solution was transferred to a glass plate and dried in a vacuum chamber. The vacuum dried tacrolimus/HPMC mixture was milled using a pestle and mortar.

Tacrolimus constituted 40% of the total solids in the finished formulation and HPMC constituted 60% of the total solids in the finished formulation.

79.1 milligrams of the vacuum dried tacrolimus/HPMC mixture was mixed with 79.6 milligrams of a liquid PEG 400/epoxy resin mixture until a homogenous paste was achieved. The PEG 400/epoxy resin mixture included 63.15% w/v Epoxy part A. 16.9% w/v Epoxy part B and 19.95% w/v PEG 400.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 10: Preparation of Composite Material and Punctal Plugs Containing Tacrolimus Via Physical Mixing

A liquid epoxy-PEG mixture having 63.9% Epoxy part A. 16.26% Epoxy part B and 19.84% PEG 4000 was formed. 40.8 milligrams HPMC powder. 60.4 milligrams tacrolimus powder and 101.3 milligrams of the Epoxy-PEG mixture were mixed using a spatula until a homogenous paste was achieved.

The paste was loaded on a stainless steel mold and the mold was put in a H-form shop press under 1 ton of pressure for 1 hour and 45 minutes. The composite was left to dry in the mold overnight at ambient conditions. The resultant plugs were then released from the mold.

### Example 11: Comparison of Spray Dried, Vacuum Dried and Physical Mixtures of Punctal Plugs Containing Tacrolimus and HPMC

Two plugs each were made from a spray dried tacrolimus and HPMC formulation, a vacuum dried tacrolimus and HPMC formulation, and a physical mixture tacrolimus and HPMC formulation. Each of the plugs were tested according to the assay described in Example 8. The results, which are an average of the release of tacrolimus from the two plugs of each formulation, are shown in FIG. 3. As depicted in FIG. 3, the spray dried formulation demonstrates a more favorable release profile because more drug is released per day than the vacuum dried formulation and the physical mixture formulation. Additionally, the spray dried formulation demonstrates a smaller burst release on the first day than the vacuum dried formulation.

### Exemplary Embodiments

Non-limiting specific embodiments are described below each of which is considered to be within the present disclosure.

Embodiment 1. A method including:
dissolving a hydrophilic polymer and an active pharmaceutical ingredient (API) in alcohol to form a hydrophilic polymer/API solution:
   where the API includes tacrolimus:
spray drying the hydrophilic polymer/API solution to form a hydrophilic polymer/API mixture;
mixing the hydrophilic polymer/API mixture with a binder to form an API paste;
curing the API paste to form a punctal plug:
   where the punctal plug is configured to release an effective amount of the API for a treatment period of 30 days to one year.

Embodiment 2. The method of embodiment 1, where the hydrophilic polymer is hydroxypropyl methylcellulose (HPMC).

Embodiment 3. The method of embodiment 1, where the binder is an epoxy resin.

Embodiment 4. The method of embodiment 1, where the effective amount of the API is at least 0.6 μg per day.

Embodiment 5. The method of embodiment 1, where the effective amount of the API measures from 10% to 30% by weight of a total amount of the API mixture.

Embodiment 6. The method of embodiment 1, where the effective amount of the API measures from 30 μg to 90 μg over the treatment period of 30 days to one year.

Embodiment 7. The method of embodiment 1, further including mixing the hydrophilic polymer/API mixture with at least one additive.

Embodiment 8. The method of embodiment 7, where the at least one additive includes kaolin or polyethylene glycol (PEG).

Embodiment 9. The method of embodiment 1, where the alcohol is ethanol.

Embodiment 10. A device including:
a spray dried composite material including:
   a hydrophilic polymer; and
   an active pharmaceutical ingredient (API), where the API includes tacrolimus; and
a binder;
where the API has a release rate of at least 0.6 μg per day for a treatment period of 30 days to one year.
where the device is a punctal plug.

Embodiment 11. The device of embodiment 10, where the hydrophilic polymer is hydroxypropyl methylcellulose (HPMC).

Embodiment 12. The device of embodiment 10, where the binder is an epoxy resin.

Embodiment 13. The device of embodiment 10, where the device includes 10% to 40% of tacrolimus by weight.

Embodiment 14. The device of embodiment 10, where the device includes 10% to 40% of the hydrophilic polymer by weight.

Embodiment 15. The device of embodiment 10, where the device includes 30% to 40% of the binder by weight.

Embodiment 16. The device of embodiment 10, further including an additive.

Embodiment 17. The device of embodiment 16, where the additive includes at least one of kaolin or polyethylene glycol (PEG).

Embodiment 18. The device of embodiment 17, where the device includes 5% to 15% of PEG by weight.

Embodiment 19. The device of embodiment 17, where the device includes 0% to 10% of kaolin by weight.

Embodiment 20. A formulation including:
a spray dried composite material including:
   a hydrophilic polymer; and
   an active pharmaceutical ingredient (API),
      where the API includes tacrolimus; and a binder;
   where the API has a release rate of at least 0.6 μg per day for a treatment period of 30 days to one year.

While a number of embodiments of the present disclosure have been describe, it is understood that these embodiments are illustrative only, and not restrictive, and that many modifications may become apparent to those of ordinary skill in the art. Many modifications and variations of the present disclosure are possible in light of the above teachings. Further still, the various steps may be carried out in any desired order (and any desired steps may be added and/or any desired steps may be eliminated). Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A method including:
dissolving a hydrophilic polymer and an active pharmaceutical ingredient (API) in alcohol to form a hydrophilic polymer/API solution;
where the API includes tacrolimus:
spray drying the hydrophilic polymer/API solution to form a hydrophilic polymer/API mixture;
mixing the hydrophilic polymer/API mixture with a binder to form an API paste;
curing the API paste to form a punctal plug:
where the punctal plug is configured to release an effective amount of the API for a treatment period of 30 days to one year.

2. The method of claim 1, where the hydrophilic polymer is hydroxypropyl methylcellulose (HPMC).

3. The method of claim 1, where the binder is an epoxy resin.

4. The method of claim 1, where the effective amount of the API is at least 0.6 μg per day.

5. The method of claim 1, where the effective amount of the API measures from 10% to 30% by weight of a total amount of the API mixture.

6. The method of claim 1, where the effective amount of the API measures from 30 μg to 90 μg over the treatment period of 30 days to one year.

7. The method of claim 1, further including mixing the hydrophilic polymer/API mixture with at least one additive.

8. The method of claim 7, where the at least one additive includes kaolin or polyethylene glycol (PEG).

9. The method of claim 1, where the alcohol is ethanol.

10. A device including:
a spray dried composite material including:
a hydrophilic polymer; and
an active pharmaceutical ingredient (API),
where the API includes tacrolimus; and
a binder;
where the API has a release rate of at least 0.6 μg per day for a treatment period of 30 days to one year.
where the device is a punctal plug.

11. The device of claim 10, where the hydrophilic polymer is hydroxypropyl methylcellulose (HPMC).

12. The device of claim 10, where the binder is an epoxy resin.

13. The device of claim 10, where the device includes 10% to 40% of tacrolimus by weight.

14. The device of claim 10, where the device includes 10% to 40% of the hydrophilic polymer by weight.

15. The device of claim 10, where the device includes 30% to 40% of the binder by weight.

16. The device of claim 10, further including an additive.

17. The device of claim 16, where the additive includes at least one of kaolin or polyethylene glycol (PEG).

18. The device of claim 17, where the device includes 5% to 15% of PEG by weight.

19. The device of claim 17, where the device includes 0% to 10% of kaolin by weight.

20. A formulation including:
a spray dried composite material including:
a hydrophilic polymer; and
an active pharmaceutical ingredient (API),
where the API includes tacrolimus; and
a binder;
where the API has a release rate of at least 0.6 μg per day for a treatment period of 30 days to one year.
